(19) United States
(12) Patent Application Publication
(10) Pub. No.: US 2023/0372683 A1
(43) Pub. Date: Nov. 23, 2023

(54) ADJUSTABLE SHUNTS WITH RESONANT CIRCUITS AND ASSOCIATED SYSTEMS AND METHODS

(71) Applicant: Shifamed Holdings, LLC, Campbell, CA (US)

(72) Inventors: Peter Andriola, Castro Valley, CA (US); Brian Fahey, Menlo Psrk, CA (US)

(21) Appl. No.: 18/247,499

(22) PCT Filed: Oct. 6, 2021
(86) PCT No.: PCT/US2021/053836

§ 371 Date: Mar. 31, 2023

Related U.S. Application Data
(60) Provisional application No. 63/088,832, filed on Oct. 7, 2020.
(60) Provisional application No. 63/089,391, filed on Oct. 8, 2020.

(51) Int. Cl. A61M 27/00

(52) U.S. Cl. CPC A61M 27/002; A61M 2205/0266; A61M 2205/368; A61M 2210/125; A61M 2205/3686

(57) ABSTRACT

The present technology is generally directed to shunting systems having shape memory actuation elements that can selectively change a geometry of a shunting element to affect the flow of fluid therethrough. In some embodiments, the shape memory actuation elements are incorporated as part of an onboard resonant circuit. Activating the resonant circuit causes current to flow through the shape memory actuation element, thereby resistively heating the shape memory actuation element.

### CROSS-REFERENCE TO RELATED APPLICATION(S)

This application claims the benefit of U.S. Provisional Application No. 63/088,832, filed Oct. 7, 2020, and U.S. Provisional Application No. 63/089,391, filed Oct. 8, 2020, both of which are incorporated by reference herein in their entireties.

### TECHNICAL FIELD

The present technology generally relates to implantable medical devices and, in particular, to implantable shunting systems and associated methods for selectively controlling fluid flow between a first body region and a second body region.

### BACKGROUND

Implantable medical devices that can be selectively activated or otherwise actuated generally require some sort of power management system. The power management system can be utilized for several types of operations. For example, the power management system can be utilized to operate electrical components (e.g., microcontrollers, sensors, etc.) on the device, and can also be utilized to drive activation or actuation of aspects of the device. To this end, some medical devices include an energy storage component (e.g., a battery, capacitor, supercapacitor, etc.) that is integral with or operably-coupled to the device. Each energy storage component is associated with different characteristics (e.g., capacity, energy density, power density, discharge rate, impedance, etc.) and therefore different components may be best suited for different types of operations.

Implantable devices may also include onboard electronics for wirelessly receiving energy and charging or recharging an energy storage device. FIG. 1, for example, is a schematic diagram of a conventional electrical circuit 100 for recharging an energy storage device (which in the illustrated embodiment is capacitor C2), which can be used to power an actively-powered actuator 106. The electrical circuit 100 can include a rectified resonant RLC circuit that generates energy in response to exposure to a magnetic and/or electric field. At least some of the energy generated in the resonant RLC circuit is directed to and stored in the capacitor C2. The stored energy can then be released in a controlled manner to power the actuator 106. For example, the circuit 100 can include a switch-mode power circuit that can control the parameters of the energy released via the capacitor C2 to maintain precise control over the power delivered to the actuator 106. However, several shortcomings exist with such conventional circuits. For example, the actuator 106 cannot be directly activated by external energy sources, which necessitates the use of one or more energy storage devices and adds cost, size, and complexity to the device. Moreover, the amount of power that may be used to drive the actuator 106 is limited by the storage capacities of the energy storage device, which is generally proportional to the size of the energy storage device. Additionally, the rate of discharge and the time-discharge characteristics of an energy storage device may not allow for the full energy storage capacity to be practically utilized (e.g., when used in an environment, for instance a fluidic environment, in which energy delivered to and/or converted by actuator 106 is rapidly transferred to surrounding media), thereby further increasing the size of energy storage device that is required to successful drive the actuator 106.

While these aforementioned shortcomings speak to the desirability of integrating larger energy storage devices, the size of the energy storage device(s) may be limited by the target anatomical location of the implantable device and/or the procedure used to implant the implantable device. As such, the storage capacities and/or discharge characteristics of the energy storage devices available for use are often sub-optimal. The consequence of this is that approaches that utilize circuits similar to those in FIG. 1 are often incapable of driving actuators in implantable medical devices, or that such approaches require the use of sub-optimal (e.g., low mass) actuators to succeed. Accordingly, a need exists for improved power management systems in actively-powered medical devices.

### BRIEF DESCRIPTION OF THE DRAWINGS

FIG. 1 is a schematic illustration of an electrical circuit for charging an implantable energy storage device.

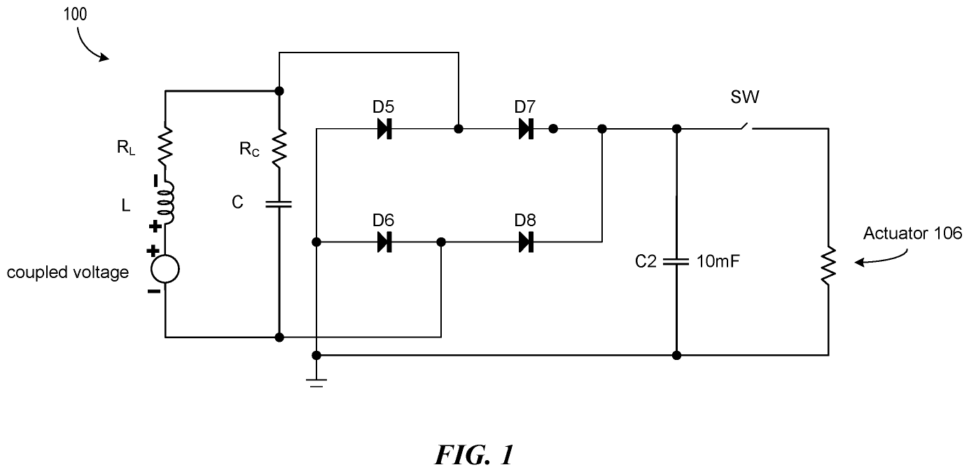

FIG. 2 is a schematic illustration of an interatrial device implanted in a heart and configured in accordance with select embodiments of the present technology.

FIG. 3 is a schematic illustration of an adjustable interatrial shunting system configured in accordance with select embodiments of the present technology.

FIG. 4 is a schematic illustration of an electrical circuit incorporating an actuation element and configured in accordance with select embodiments of the present technology.

FIG. 5 is a flowchart of a method for deploying an adjustable shunting system in accordance with embodiments of the present technology.

### DETAILED DESCRIPTION

The present technology is generally directed to adjustable shunting systems having shape memory actuation elements that can selectively change a geometry of a shunting element to affect the flow of fluid therethrough. In some embodiments, the shape memory actuation elements are incorporated directly into an onboard resonant circuit. Activating the resonant circuit (e.g., through externally applied energy, such as an externally generated magnetic field) causes current to flow through the shape memory actuation element, thereby resistively heating the shape memory actuation element. Heating the shape memory actuation element above its transition temperature can induce a change in material state in the shape memory actuation element, which may induce a geometric change in the actuation element that can drive a corresponding geometric change in the shunting element.

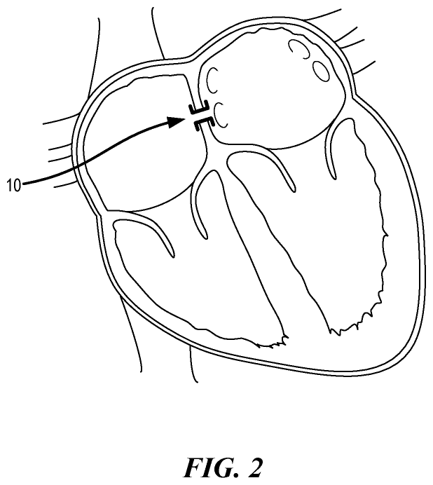

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 2-4.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

As used herein, the terms “interatrial device,” “interatrial shunt device,” “IAD,” “IASD,” “interatrial shunt,” and “shunt” are used interchangeably to refer to a device that, in at least one configuration, includes a shunting element that provides a blood flow between a first region (e.g., a left atrium of a heart) and a second region (e.g., a right atrium or coronary sinus of the heart) of a patient. Although described in terms of a shunt between the atria, namely the left and right atria, one will appreciate that the technology may be applied equally to devices positioned between other chambers and passages of the heart, or between other parts of the cardiovascular system. For example, any of the shunts described herein, including those referred to as “interatrial,” may be nevertheless used and/or modified to shunt blood between the left atrium (“LA”) and the coronary sinus, or between the right pulmonary vein and the superior vena cava. Moreover, while the disclosure herein primarily describes shunting blood from the LA to the right atrium (“RA”), the present technology can be readily adapted to shunt blood from the RA to the LA to treat certain conditions, such as pulmonary hypertension. For example, mirror images of embodiments, or in some cases identical embodiments, used to shunt blood from the LA to the RA can be used to shunt blood from the RA to the LA in certain patients. Moreover, while certain embodiments herein are described in the context of heart failure treatment, any of the embodiments herein, including those referred to as interatrial shunts, may nevertheless be used and/or modified to treat other diseases or conditions, including other diseases or conditions of other body regions. For example, the systems described herein can be used to treat diseases characterized by increased pressure and/or fluid build-up, including but not limited to glaucoma, pulmonary failure, renal failure, hydrocephalus, and the like.

The headings provided herein are for convenience only and do not interpret the scope or meaning of the claimed present technology.

### A. Interatrial Shunts for Treatment of Heart Failure

Heart failure (“HF”) can be classified into one of at least two categories based upon the ejection fraction a patient experiences: (1) heart failure with preserved ejection fraction (“HFpEF”), historically referred to as diastolic heart failure or (2) heart failure with reduced ejection fraction (“HFrEF”), historically referred to as systolic heart failure. One definition of HFrEF is a left ventricular ejection fraction lower than 35%-40%. Though related, the underlying pathophysiology and the treatment regimens for each heart failure classification may vary considerably. For example, while there are established pharmaceutical therapies that can help treat the symptoms of HFrEF, and at times slow or reverse the progression of the disease, there are limited available pharmaceutical therapies for HFpEF with only questionable efficacy.

In heart failure patients, abnormal function in the left ventricle (“LV”) leads to pressure build-up in the LA. This leads directly to higher pressures in the pulmonary venous system, which feeds the LA. Elevated pulmonary venous pressures push fluid out of capillaries and into the lungs. This fluid build-up leads to pulmonary congestion and many of the symptoms of heart failure, including shortness of breath and signs of exertion with even mild physical activity. Risk factors for HF include renal dysfunction, hypertension, hyperlipidemia, diabetes, smoking, obesity, old age, and obstructive sleep apnea. HF patients can have increased stiffness of the LV which causes a decrease in left ventricular relaxation during diastole resulting in increased pressure and inadequate filling of the ventricle. HF patients may also have an increased risk for atrial fibrillation and pulmonary hypertension, and typically have other comorbidities that can complicate treatment options.

Interatrial shunts have recently been proposed as a way to reduce elevated left atrial pressure, and this emerging class of cardiovascular therapeutic interventions has been demonstrated to have significant clinical promise. FIG. 2 shows the placement of a shunt in the septal wall between the LA and RA. Most interatrial shunts (e.g., shunt 10) involve creating a hole or inserting a structure with a lumen into the atrial septal wall, thereby creating a fluid communication pathway between the LA and the RA. As such, elevated left atrial pressure may be partially relieved by unloading the LA into the RA. In early clinical trials, this approach has been shown to improve symptoms of heart failure.

One challenge with many conventional interatrial shunts is determining the most appropriate size and shape of the shunt lumen. A lumen that is too small may not adequately unload the LA and relieve symptoms; a lumen that is too large may overload the RA and right-heart more generally, creating new problems for the patient. Moreover, the relationship between pressure reduction and clinical outcomes and the degree of pressure reduction required for optimized outcomes is still not fully understood, in part because the pathophysiology for HFpEF (and to a lesser extent, HFrEF) is not completely understood. As such, clinicians are forced to take a best guess at selecting the appropriately sized shunt (based on limited clinical evidence) and generally cannot adjust the sizing over time. Further, clinicians must select the size of the shunt based on general factors (e.g., the size of the patient's anatomical structures, the patient's hemodynamic measurements taken at one snapshot in time, etc.) and/or the design of available devices rather than the individual patient's health and anticipated response. With many such traditional devices, the clinician does not have the ability to adjust or titrate the therapy once the device is implanted, for example, in response to changing patient conditions such as progression of disease. By contrast, interatrial shunting systems configured in accordance with embodiments of the present technology allow a clinician to select the size—perioperatively or post-implant—based on the patient.

### B. Adjustable Shunting Systems

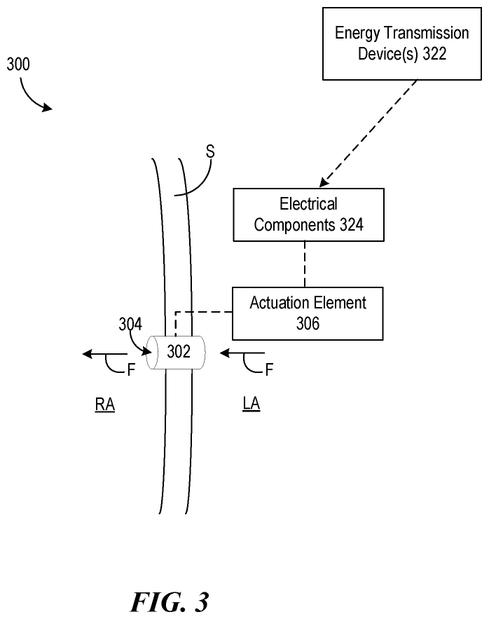

FIG. 3 is a schematic illustration of an adjustable shunting system 300 (“system 300”) configured in accordance with an embodiment of the present technology. The system 300 includes a shunting element 302 defining a lumen 304 therethrough. When implanted across the septal wall S, the system 300 can fluidly connect the LA and the RA via the lumen 304. Accordingly, when the shunting element 302 is implanted in the septal wall, blood can flow from the LA to the RA via the lumen 304 (as shown by arrows F). The shunting element 302 can include additional features not shown in FIG. 2, such as a frame, anchors, membrane, or the like. For example, the shunting element 302 can include features such as those described in International Patent Application No. PCT/US2020/049996, the disclosure of which is incorporated by reference in its entirety.

The system 300 can further include an actuation element 306 configured to selectively change a geometry (size, shape, etc.) and/or other characteristic of the shunting element 302 to selectively modulate the flow of fluid through the lumen 304. For example, the actuation element 306 can be configured to selectively increase a diameter (e.g., an orifice diameter, hydraulic diameter, etc.) of the lumen 304 and/or selectively decrease a diameter (e.g., an orifice diameter, hydraulic diameter, etc.) of the lumen 304 in response to an input. In other embodiments, the actuation element 306 is configured to otherwise affect a shape and/or geometry of the lumen 304. Accordingly, the actuation element 306 can be coupled to the shunting element 302 and/or can be included within the shunting element 302. In some embodiments, for example, the actuation element 306 is part of the shunting element 302 and at least partially defines the lumen 304. In other embodiments, the actuation element 306 is spaced apart from but operably coupled to the shunting element 302.

In some embodiments, at least a portion of the actuation element 306 comprises a shape memory material, such as a shape memory metal or alloy (e.g., nitinol, including nitinol-based alloys), a shape memory polymer, or a pH-based shape memory material. In embodiments in which the actuation element is composed of a shape memory material (which may be referred to herein as a “shape memory actuation element”), the shape memory actuation element can be configured to change in geometry (e.g., transform between a first configuration and a second configuration) in response to a stimulus (e.g., heat or mechanical loading). For example, in some embodiments the shape memory actuation element is deformed relative to its preferred geometry (e.g., manufactured geometry, original geometry, heat set geometry, etc.) when it is in a first material state (e.g., martensitic material state or R-phase material state). When the deformed shape memory element is heated above its transition temperature (which in some embodiments is a temperature greater than body temperature), the shape memory actuation element transitions to a second material state (e.g., R-phase material state or austenitic material state), which can cause the shape memory actuation element to move toward its preferred geometry. The movement of the actuation element from the deformed position toward its preferred geometry can adjust the geometry of the lumen 304, as described above. Additional aspects of adjusting an interatrial shunt using shape memory actuation elements, including various adjustable interatrial shunts incorporating shape memory actuation elements, are described in International Application No. PCT/US2020/049996, previously incorporated by reference herein.

The system 300 can further include an energy transmission device(s) 322 for delivering energy (e.g., power) to the implanted components (e.g., the actuation element 306 and/or the implanted electrical components 324, described below) of the system 300. The energy transmission device(s) 322 can include any device or system external to the patient's body that is capable of wirelessly transmitting energy to an implanted component. For example, an energy transmission device 322 can be configured to transmit radiofrequency (RF) energy, microwave frequency energy, other forms of electromagnetic energy, ultrasonic energy, thermal energy, or other types of energy in accordance with techniques known to those of skill in the art. In some embodiments, the energy transmission device 322 may deliver energy having a frequency in a range of between about 1 MHz and about 1 GHz (e.g., 1 MHz, 2 MHz, 3 MHz, 10 MHz, 100 MHz, 500 MHz, etc.), although other frequencies are possible. In some embodiments, the energy transmission device may generate an electric and/or magnetic field directed toward the implanted aspects of the system 300.

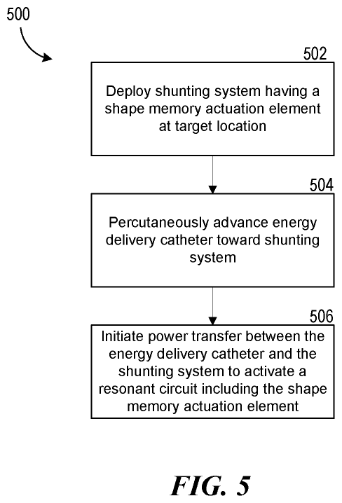

In some embodiments, the energy transmission device(s) 322 can include one or more devices configured to be positioned at least temporarily within the patient's body (e.g., an energy delivery catheter configured to be navigated proximate to the system 300 during a procedure). For example, the energy transmission device 322 can be advanced percutaneously until a transmitter coil on a distal end of the energy transmission device 322 is proximate to (e.g., within 5 cm, within 4 cm, within 3 cm, within 2 cm, within 1 cm, etc.) the one or more implanted portions of the system 300, such as the actuation element 306. Once positioned proximate to the actuation element 306 or another target component, the energy transmission device 322 can generate an electromagnetic field for activating the actuation element 306. Of note, and similar to the electrical coupling achieved when using an energy transmission device 322 positioned external to the patient, the energy delivery catheter need not be positioned in direct contact with any implanted portion of the system 300, such as the actuation element 306. However, use of an energy delivery catheter instead of or in addition to a non-invasive energy transmission device positioned external to the patient's body is expected to be useful in embodiments in which a greater amount of energy is required because the coupling efficiency when using a transmitter positioned proximate to the target is expected to be substantially greater (e.g., at least about 100% greater, at least about 1,000% greater, etc.) than when using a transmitter that remains external to the patient's body. A representative embodiment in which an invasive approach using an energy delivery catheter is particularly useful is described in greater detail below with reference to FIG. 5.

The system 300 can further include electrical components 324 implanted with the shunting element 302 and electrically coupled together to form electrical circuits (e.g., RLC circuits, resonant circuits, etc.). The electrical components 324 can include, for example, conventional electrical components found in electrical circuits, such as resistors, capacitors, and inductors. In some embodiments, the inductors can be wire coils or other filaments capable of coupling to externally produced electromagnetic fields. For example, in some embodiments the inductor is a first wire coil (not shown) having a self-inductance L. The energy transmission device 322 can have a second wire coil (not shown) configured to remain external to the patient (or at least remain spaced apart from the electrical components 324, such as being positioned on an energy delivery catheter), and coupled to the first wire coil through a mutual-inductance M. The self-inductance L of the first wire coil acts as the inductor in the electrical circuit, while the mutual-inductance M of the first and second wire coils acts to transfer energy and/or power from an externally generated magnetic field to the first wire coil. In some embodiments, the self-inductance L can be between about 0.1 pH and about 10 pH, a ratio of the mutual-inductance M to the self-inductance L (e.g., M/L) can be less than about 0.10, and a ratio of the diameter of the first wire coil to a diameter of the second wire coil can be between about 0.01 to 1.0. In some embodiments, the first wire coil may be implemented with one or more turns of formed wire, conductive patterns printed on a non-conducting single- or multi-layer substrate, or another conductive structure formed so as to encircle magnetic flux produced by the energy transmission device 322.

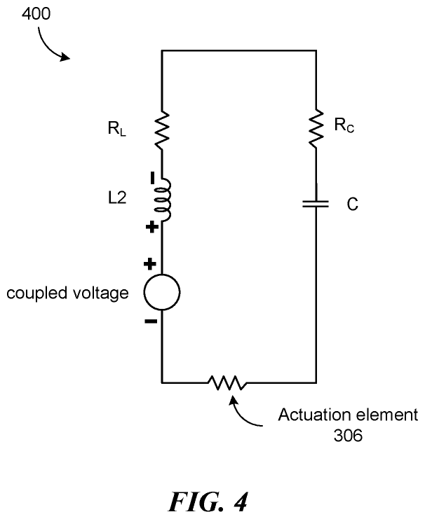

As described in detail with respect to FIG. 3, the electrical circuits formed by the electrical components 324 can receive energy and/or power from the energy transmission device 322. For example, in some embodiments, the energy transmission device 322 generates an electromagnetic field, and the electrical components 324 generate an electrical current in response to being exposed to the electromagnetic field. The current generated by the electrical components 324 can flow through and directly provide power to (e.g., resistively heat) the actuation element 306. For example, as described in greater detail with respect to FIG. 4, the actuation element 306 can be incorporated into the electrical circuit formed by the electrical components 324 such that the energy transmission device 322 can directly power the actuation element 306 by generating a current in the electrical circuit that flows through and resistively heats the actuation element 306.

FIG. 4 is a circuit diagram of an exemplary electrical circuit formed via the electrical components 324 (FIG. 3) and used to power the actuation element 306 in accordance with an embodiment of the present technology. In particular, FIG. 4 illustrates a resonant circuit 400 configured to be powered via the energy transmission device 322. Notably, because the actuation element 306 can be powered through resistive heating and does not require a specific electrical waveform like many conventional actuators, including electric motors, the actuation element 306 can be directly incorporated into the resonant circuit 400. For example, in the illustrated embodiment, the actuation element 306 is coupled in series with the other electrical components of the resonant circuit 400. In other embodiments, the actuation element 306 can be coupled in parallel with other electrical components of the resonant circuit 400, or in other suitable configurations. When the resonant circuit 400 is energized (e.g., via the external energy transmission device(s) 322—FIG. 3), current flows through the actuation element 306, resistively heating the actuation element 306. In embodiments in which the actuation element 306 is composed of a shape memory material, this resistive heating may heat the shape memory actuation element above its transition temperature and drive the phase transformation that induces a geometry change in the lumen 304, as described in detail above with respect to FIG. 3.

The resonant circuit 400 can be characterized by a quality factor Q, which is the ratio of the energy stored in the circuit to the energy dissipated per radian of resonant oscillation and is defined by the following equation:

$$Q = \frac{2 π fL}{{R}_{L} + {R}_{C} + R 1}$$

In the above equation, f equals the resonant frequency, L is the inductance of the inductor, R1 equals the resistance of the actuation element 306, R_{L} is the resistance of the inductor, and R_{C} is the resistance of the capacitor. In some embodiments, R_{L} has a value between about 0.1 Ohm and about 2 Ohms, R_{C} has a value between about 0.01 Ohms and 0.05 Ohms, and R1 has a value between about 0.5 Ohms and about 10 Ohms, although values outside the foregoing ranges are possible and within the scope of the present technology.

The resonant circuit 400 can be designed to optimize or otherwise enhance the functionality of the system 300. For example, contrary to conventional resonant circuits which typically attempt to maximize the quality factor Q by minimizing the power dissipated in the circuit, the resonant circuit 400 can be designed to have intentional “power loss.” In particular, the actuation element 306 is designed to increase the power dissipated in the circuit as current flows through it (thereby lowering the quality factor Q), since the power dissipation caused by the current flowing through the actuation element 306 drives the temperature change that induces the geometric change of the actuation element 306. In some embodiments, for example, the resonant circuit 400 may have a quality factor Q less than about 100, such as between about 10 and about 100. In contrast, conventional wireless power transfer circuits generally have a quality factor Q greater than 100. The power dissipation in the actuation element 306 is generally maximized when the resistance of the actuation element 306 is equal or at least approximately equal to R_{L}+R_{C}. Accordingly, in some embodiments, the resistance of the actuation element 306 is equal or substantially equal to the sum of R_{L} and R_{C}, such that the ratio of the resistance of the actuation element 306 to the sum of the resistance of R_{L} and R_{C} is between 2.0 and 0.5, such as 2.0, 1.8, 1.6, 1.4, 1.2, 1.1, 1.0, 0.9, 0.8, 0.7, 0.6, or 0.5.

The resonant circuit 400 can also be designed such that the actuation element 306 has a greater power dissipation density than the other electrical components (e.g., the inductor, the capacitor, etc.) when current flows through the circuit 400. For example, the other electrical components can (each) have a surface area that is greater than a surface area of the actuation element 306. In some embodiments, this may be accomplished by designing the other electrical components to have a greater length than the actuation element 306, although other configurations are also possible (e.g., the other electrical components can have a larger circumference than the actuation element 306). As a result of the actuation element 306 having a relatively smaller surface area, the power dissipation density in the actuation element 306 is greater than the power dissipation density in the other electrical components. This beneficially causes the actuation element 306 to be heated to a greater degree than the other electrical components as current flows through the circuit 400.

As described above, incorporating the actuation element 306 into the resonant circuit 400 generally provides additional resistance (and thus greater power dissipation) within the resonant circuit 400. However, such additional resistance is generally low enough to avoid disrupting the circuit resonance.

Incorporating the actuation element 306 into the resonant circuit 400 is expected to provide several advantages. For example, incorporating the actuation element 306 into the resonant circuit 400 enables the actuation element 306 to be directly heated, and thus directly activated, using an energy transmission device external to the permanently implanted portion of system 300 (e.g., energy transmission device(s) 322—FIG. 2). In contrast, if the actuation element 306 were not incorporated into the resonant circuit 400, the system 300 would require an energy storage component (e.g., a supercapacitor that could receive and store energy form the resonant circuit) to selectively release energy to heat the actuation element. Such energy storage elements that have a size and/or shape suitable for implantation and/or delivery (e.g., percutaneous delivery via a catheterization procedure) into a patient's heart generally have limited energy storage capacities and/or discharge characteristics, which can limit the ability to actuate the actuation element.

In contrast, by not relying on an implanted energy storage component, the present technology is not limited by the energy storage capacities or other properties of the energy storage component, and instead relies on external energy transmission devices (which can have a relatively unlimited supply of energy) to directly activate the actuation element 306. Accordingly, in some embodiments the present technology is expected to provide adjustable shunting systems that do not require (permanently) implanted energy storage components to actuate the actuation elements. However, in some embodiments, the adjustable shunting systems may nevertheless include implanted energy storage components for powering other aspects of the system (e.g., sensors) and/or for augmenting the power provided by the external energy transmission devices.

Another expected advantage of incorporating the actuation element 306 into the resonant circuit 400 rather than relying on a separate energy storage component is that, even when energy is continuously delivered, the system 300 does not generate direct current (DC) when energizing the actuation element 306 (which would be the case if an energy storage component such as a supercapacitor were utilized). Without being bound by theory, this is expected to reduce/obviate the risk of electric shock from such energy transmission.

Alternatively or additionally, the implantable devices and systems described herein can include a resonant antenna or cavity integrated with the shape memory actuation element. In such embodiments, the resonant antenna or cavity can be configured to operate at a frequency above about 100 MHz. In response to the resonant circuits in the antenna or cavity being powered, current can flow through and heat the shape memory actuation element, as described above. In other embodiments, the implantable devices and systems described herein can include a piezoelectric acoustic resonator configured to receive energy from an externally positioned ultrasound source. The piezoelectric acoustic resonator can convert the received energy into an electrical current. The piezoelectric acoustic resonator can also be operably coupled (e.g., coupled in series) with a shape memory actuation element such that the electrical current flows through and resistively heats the shape memory actuation element.

As set forth above, the present technology can be utilized to non-invasively power an actuation element (e.g., using an energy transmission device that remains external to the patient) or to invasively power an actuation element (e.g., using an energy transmission device that is advanced percutaneously toward the implanted device). In some embodiments, a system is configured to enable a user (e.g., physician) to opt to power the device non-invasively or invasively depending on a number of factors, including the environments/setting in which therapy occurs (e.g., availability of catheter-lab), energy requirements, timing requirements, patient factors (e.g., risk factors) or the like. Without being bound by theory, invasively powering the actuation element is expected to provide more efficient energy coupling between the energy transmission device (e.g., expected energy coupling between the transmitter and receiver of about 10-30%) compared to non-invasively powering the actuation element (e.g., expected energy coupling between the transmitter and receiver of generally less than 1%). Of course, non-invasively powering the actuation element may be favorable in some circumstances in which activation requires lower amounts of total energy transfer because it is less complex/invasive.

FIG. 5 is a flowchart of a method 500 for deploying an adjustable shunting system having a shape memory actuation element and a shunting element (e.g., the system 300, the shunting element 302, and the actuation element 306 of FIG. 3) in accordance with embodiments of the present technology. The method 500 can begin at step 502 by deploying a shunting system having a shape memory actuation element at a target location, such as across a septal wall of a heart. For example, step 502 can include percutaneously advancing a shunt delivery catheter carrying the shunting system to the target location, deploying the shunting system from the shunt delivery catheter, and retracting the shunt delivery catheter. In such embodiments, the shunting system is generally provided in a collapsed configuration within the catheter before deployment. Upon deployment from the catheter, the shunting system typically expands toward its deployed configuration. However, certain components, such as the shape memory actuation element, may remain at least partially deformed (e.g., crumpled) even after deployment from the catheter. In some embodiments, the shape memory actuation element is deformed to an extent that affects its function, necessitating additional recovery of the shape memory actuation element.

The method 500 can continue at step 504 by percutaneously advancing an energy delivery catheter toward the shunting system. The energy delivery catheter can be advanced until a transmitting coil on the energy delivery catheter is positioned within 5 cm, within 4 cm, within 3 cm, within 2 cm, and/or within 1 cm of a target component (e.g., the deformed actuation element) of the shunting system. Once the coil on the energy delivery catheter is in position, the method 500 can continue at step 506 by initiating power transfer between the energy deliver catheter and shunting system to activate a resonant circuit including the shape memory actuation element. This can include emitting an electromagnetic field from the transmitting coil and inducing an electrical current in the resonant circuit in response to the electromagnetic field, as described with reference to FIGS. 3 and 4. This causes electrical current to flow through, and resistively heat, the shape memory actuation element. The shape memory actuation element can be heated above a transition temperature such that it transitions to a relatively stiffer material state (e.g., austenitic), which causes the shape memory actuation element to move toward and recover its preferred geometry.

Although steps 504 and 506 describe utilizing an invasive energy delivery device to recover the shape of the shape memory actuation following deployment of the shunting system, in some embodiments steps 504 can be omitted and step 506 can be performed using an energy transmission device that remains external to the patient's body. However, in some embodiments, the invasive approach described with steps 504 and 506 is preferred because the amount of energy required to at least initially recover the shape memory actuation element is great enough that using a non-invasive approach is impractical and/or not efficient. Once the shape memory actuation element has been recovered, however, non-invasive approaches can be used to make further adjustments to the shape memory actuation element.

As one of skill in the art will appreciate from the disclosure herein, various components of the interatrial shunting systems described above can be omitted without deviating from the scope of the present technology. Likewise, additional components not explicitly described above may be added to the interatrial shunting systems without deviating from the scope of the present technology. Moreover, the electrical circuits described herein can be incorporated into other types of implantable medical devices beyond cardiac shunts. Accordingly, the present technology is not limited to the configurations expressly identified herein, but rather encompasses variations and alterations of the described systems.

### Examples

Several aspects of the present technology are set forth in the following examples:
1. An implantable medical device, the device comprising:
an actuation element composed of a shape memory material and having a preferred geometry, wherein, when the actuation element is deformed relative to its preferred geometry and is heated above a transition temperature, the actuation element is configured to move toward its preferred geometry; and
one or more electrical components configured to generate a current when exposed to an electromagnetic field, wherein the one or more electrical components form a resonant circuit that includes the actuation element, and wherein the resonant circuit is configured such that current flows through and resistively heats the actuation element when the one or more electrical components generate the current.
2. The device of example 1 wherein the one or more electrical components are configured to generate a current when exposed to an electromagnetic field generated by an energy source positioned external to the patient.
3. The device of example 1 wherein the one or more electrical components are configured to generate a current when exposed to an electromagnetic field generated by an energy source positioned within the patient and spaced apart from the one or more electrical components.
4. The device of example 1 wherein the one or more electrical components are configured to generate a current in response to delivery of radiofrequency (RF) and/or microwave energy.
5. The device of any of examples 1-4 wherein the resonant circuit is an RLC circuit.
6. The device of any of examples 1-5 wherein a ratio between a first resistance provided by the actuation element and a second resistance provided by the one or more electrical components is between about 2:1 and 0.5:1.
7. The device of any of examples 1-6 wherein the actuation element has a first surface area, and wherein the one or more electrical components have a second surface area greater than the first surface area.
8. The device of example 7 wherein, when current flows through the one or more electrical components and the actuation element, a first power dissipation density in the actuation element is greater than a second power dissipation density in the one or more electrical components by virtue of the second surface area being greater than the first surface area.
9. The device of example 7 wherein the actuation element has a first length, and wherein the one or more electrical components have a second length greater than the first length.
10. The device of any of examples 1-9 wherein the actuation element has a first resistance and the one or more electrical components collectively have a second resistance, and wherein the first resistance is approximately the same as the second resistance.
11. The device of any of examples 1-10 wherein the resonant circuit is configured to dissipate power as the current flows through the actuation element.
12. The device of any of examples 1-11 wherein the resonant circuit has a quality factor of less than 100.
13. The device of any of examples 1-12 wherein the actuation element is in series with the one or more electrical components.
14. The device of any of examples 1-13 wherein the shape memory material includes an alloy comprising one or more of nickel, titanium, and copper.
15. The device of any of examples 1-14 wherein, when implanted in a human patient, the device is configured to shunt fluid between a first body region and a second body region.
16. The device of example 15, further comprising a shunting element having a lumen extending therethrough and configured such that, when the shunting element is implanted in the patient, the lumen fluidly connects the first body region and the second body region, wherein the actuation element is configured to adjust a geometry of the lumen.
17. An electrical circuit for use with an implantable medical device, the electrical circuit comprising:
one or more electrical components configured to generate a current when exposed to an electromagnetic field; and
a shape memory actuation element integral to a circuit with the one or more electrical components,
wherein the current generated by the one or more electrical components in response to being exposed to the electromagnetic field flows through and resistively heats the shape memory actuation element.
18. The electrical circuit of example 17 wherein the circuit is a resonant circuit.
19. The electrical circuit of example 17 wherein the circuit is an RLC circuit.
20. The electrical circuit of any of examples 17-19 wherein a ratio between a first resistance provided by the shape memory actuation element and a second resistance provided by the one or more electrical components is between about 2.0:1 and 0.5:1.
21. The electrical circuit of any of examples 17-20 wherein the shape memory actuation element has a first surface area, and wherein the one or more electrical components have a second surface area greater than the first surface area.
22. The electrical circuit of example 21 wherein, when current flows through the one or more electrical components and the actuation element, a first power dissipation density in the actuation element is greater than a second power dissipation density in the one or more electrical components by virtue of the second surface area being greater than the first surface area.
23. The electrical circuit of any of examples 21 wherein the actuation element has a first length, and wherein the one or more electrical components have a second length greater than the first length.
24. The electrical circuit of any of examples 17-23 wherein the shape memory actuation element has a first resistance and the one or more electrical components collectively have a second resistance, and wherein the first resistance is the same as the second resistance.
25. The electrical circuit of any of examples 17-24 wherein the circuit is configured to dissipate power as the current flows through the shape memory actuation element.
26. The electrical circuit of any of examples 17-25 wherein the circuit has a quality factor of less than 100.
27. The electrical circuit of any of examples 17-26 wherein the shape memory actuation element is in series with the one or more electrical components.
28. The electrical circuit of any of examples 17-27 wherein the shape memory actuation element has a transition temperature, and wherein the circuit is configured such that, when the one or more electrical components are exposed to the electromagnetic field, the generated current resistively heats at least a portion of the shape memory actuation element above its transition temperature.
29. A method for controlling a medical device implanted in a patient, the method comprising:
directing energy toward one or more electrical components implanted in the patient, wherein the electrical components form a resonant circuit that includes an actuation element operably coupled to the implanted medical device; and
in response to the energy, automatically generating a current in the resonant circuit, wherein the current flows through and resistively heats the actuation element.
30. The method of example 29 wherein directing the energy toward the one or more electrical components includes directing energy from an energy source positioned external to the patient.
31. The method of example 29 wherein directing the energy toward the one or more electrical components includes directing energy from an energy source temporarily positioned within the patient but spaced apart from the one or more electrical components.
32. The method of any of examples 29-31 wherein directing the energy toward the one or more electrical components includes generating an electromagnetic field around the one or more electrical components.
33. The method of any of examples 29-32 wherein directing the energy toward the one or more electrical components includes directing RF or microwave energy toward the one or more electrical components.
34. The method of any of examples 29-33 wherein the actuation element is composed of a shape memory material, and wherein resistively heating the actuation element heats the actuation element above a transition temperature, wherein the transition temperature is a temperature greater than body temperature.
35. The method of example 34 wherein heating the actuation element above the transition temperature transforms the actuation element from a first configuration in which it is deformed relative to a preferred geometry to and/or toward a second configuration in which it assumes its preferred geometry.
36. The method of example 35 wherein moving the actuation element from the first configuration toward the second configuration controls one or more operations of the implanted medical device.
37. The method of example 36 wherein the implanted medical device is a shunt fluidly connecting a first body region and a second body region, and wherein moving the actuation element from the first configuration toward the second configuration adjusts a geometry of the shunt.
38. A method for deploying an adjustable shunting system having a shape memory actuation element, the method comprising:
deploying the adjustable shunting system that incudes the shape memory actuation element at a target location within the patient, wherein the shape memory actuation element is deformed relative to a preferred geometry following deployment of the adjustable shunting system;
percutaneously advancing an energy delivery catheter toward the shunting system until the energy delivery catheter is proximate the adjustable shunting system; and
initiating a power transfer between the energy delivery catheter and the shunting system to induce a current in a resonant circuit that includes the shape memory actuation element,
wherein the current resistively heats the shape memory actuation element.
39. The method of example 38 wherein the energy delivery catheter is a first catheter, and wherein deploying the adjustable shunting system at the target location includes percutaneously advancing a second catheter different than the first catheter toward the target location, the second catheter carrying the adjustable shunting system.
40. The method of example 38 or 39 wherein percutaneously advancing the energy delivery catheter until the energy delivery catheter is proximate the adjustable shunting system includes positioning a transmitter coil carried by the energy delivery catheter within 5 cm of the adjustable shunting system.
41. The method of example 40 wherein percutaneously advancing the energy delivery catheter until the energy delivery catheter is proximate the adjustable shunting system includes positioning the transmitter coil within 2 cm of the adjustable shunting system.
42. The method of example 40 wherein the transmitter coil does not contact the adjustable shunting system.
43. The method of any of examples 38-42 wherein the current resistively heats the shape memory actuation element above a transition temperature and causes the shape memory actuation element to move from the deformed configuration toward its preferred geometry.

### CONCLUSION

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the LA and RA, the LV and the right ventricle (RV), or the LA and the coronary sinus, it should be appreciated that similar embodiments could be utilized for shunts between other chambers of heart or for shunts in other regions of the body.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An implantable medical device, the device comprising:
an actuation element composed of a shape memory material and having a preferred geometry, wherein, when the actuation element is deformed relative to its preferred geometry and is heated above a transition temperature, the actuation element is configured to move toward its preferred geometry; and
one or more electrical components configured to generate a current when exposed to an electromagnetic field, wherein the one or more electrical components form a resonant circuit that includes the actuation element, and wherein the resonant circuit is configured such that current flows through and resistively heats the actuation element when the one or more electrical components generate the current.

2. The device of claim 1 wherein the one or more electrical components are configured to generate a current when exposed to an electromagnetic field generated by an energy source positioned external to the patient.

3. The device of claim 1 wherein the one or more electrical components are configured to generate a current when exposed to an electromagnetic field generated by an energy source positioned within the patient and spaced apart from the one or more electrical components.

4. The device of claim 1 wherein the one or more electrical components are configured to generate a current in response to delivery of radiofrequency (RF) and/or microwave energy.

5. The device of claim 1 wherein the resonant circuit is an RLC circuit.

6. The device of claim 1 wherein a ratio between a first resistance provided by the actuation element and a second resistance provided by the one or more electrical components is between about 2:1 and 0.5:1.

7. The device of claim 6 wherein the actuation element has a first surface area, and wherein the one or more electrical components have a second surface area greater than the first surface area.

8. The device of claim 7 wherein, when current flows through the one or more electrical components and the actuation element, a first power dissipation density in the actuation element is greater than a second power dissipation density in the one or more electrical components by virtue of the second surface area being greater than the first surface area.

9. The device of claim 7 wherein the actuation element has a first length, and wherein the one or more electrical components have a second length greater than the first length.

10. The device of claim 1 wherein the actuation element has a first resistance and the one or more electrical components collectively have a second resistance, and wherein the first resistance is approximately the same as the second resistance.

11. The device of claim 1 wherein the resonant circuit is configured to dissipate power as the current flows through the actuation element.

12. The device of claim 1 wherein the resonant circuit has a quality factor of less than 100.

13. The device of claim 1 wherein the actuation element is in series with the one or more electrical components.

14. The device of claim 1 wherein the shape memory material includes an alloy comprising one or more of nickel, titanium, and copper.

15. The device of claim 1 wherein, when implanted in a human patient, the device is configured to shunt fluid between a first body region and a second body region.

16. The device of claim 15, further comprising a shunting element having a lumen extending therethrough and configured such that, when the shunting element is implanted in the patient, the lumen fluidly connects the first body region and the second body region, wherein the actuation element is configured to adjust a geometry of the lumen.

17. An electrical circuit for use with an implantable medical device, the electrical circuit comprising:
one or more electrical components configured to generate a current when exposed to an electromagnetic field; and
a shape memory actuation element integral to a circuit with the one or more electrical components,
wherein the current generated by the one or more electrical components in response to being exposed to the electromagnetic field flows through and resistively heats the shape memory actuation element.

18. The electrical circuit of claim 17 wherein the circuit is a resonant circuit.

19. The electrical circuit of claim 17 wherein the circuit is an RLC circuit.

20. The electrical circuit of claim 17 wherein a ratio between a first resistance provided by the shape memory actuation element and a second resistance provided by the one or more electrical components is between about 2.0:1 and 0.5:1.

21. The electrical circuit of claim 20 wherein the shape memory actuation element has a first surface area, and wherein the one or more electrical components have a second surface area greater than the first surface area.

22. The electrical circuit of claim 21 wherein, when current flows through the one or more electrical components and the actuation element, a first power dissipation density in the actuation element is greater than a second power dissipation density in the one or more electrical components by virtue of the second surface area being greater than the first surface area.

23. The electrical circuit of claim 21 wherein the actuation element has a first length, and wherein the one or more electrical components have a second length greater than the first length.

24. The electrical circuit of claim 17 wherein the shape memory actuation element has a first resistance and the one or more electrical components collectively have a second resistance, and wherein the first resistance is the same as the second resistance.

25. The electrical circuit of claim 17 wherein the circuit is configured to dissipate power as the current flows through the shape memory actuation element.

26. The electrical circuit of claim 17 wherein the circuit has a quality factor of less than 100.

27. The electrical circuit of claim 17 wherein the shape memory actuation element is in series with the one or more electrical components.

28. The electrical circuit of claim 17 wherein the shape memory actuation element has a transition temperature, and wherein the circuit is configured such that, when the one or more electrical components are exposed to the electromagnetic field, the generated current resistively heats at least a portion of the shape memory actuation element above its transition temperature.

29. A method for controlling a medical device implanted in a patient, the method comprising:
directing energy toward one or more electrical components implanted in the patient, wherein the electrical components form a resonant circuit that includes an actuation element operably coupled to the implanted medical device; and
in response to the energy, automatically generating a current in the resonant circuit, wherein the current flows through and resistively heats the actuation element.

30. The method of claim 29 wherein directing the energy toward the one or more electrical components includes directing energy from an energy source positioned external to the patient.

31. The method of claim 29 wherein directing the energy toward the one or more electrical components includes directing energy from an energy source temporarily positioned within the patient but spaced apart from the one or more electrical components.

32. The method of claim 29 wherein directing the energy toward the one or more electrical components includes generating an electromagnetic field around the one or more electrical components.

33. The method of claim 29 wherein directing the energy toward the one or more electrical components includes directing RF or microwave energy toward the one or more electrical components.

34. The method of claim 29 wherein the actuation element is composed of a shape memory material, and wherein resistively heating the actuation element heats the actuation element above a transition temperature, wherein the transition temperature is a temperature greater than body temperature.

35. The method of claim 34 wherein heating the actuation element above the transition temperature transforms the actuation element from a first configuration in which it is deformed relative to a preferred geometry to and/or toward a second configuration in which it assumes its preferred geometry.

36. The method of claim 35 wherein moving the actuation element from the first configuration toward the second configuration controls one or more operations of the implanted medical device.

37. The method of claim 36 wherein the implanted medical device is a shunt fluidly connecting a first body region and a second body region, and wherein moving the actuation element from the first configuration toward the second configuration adjusts a geometry of the shunt.

38. A method for deploying an adjustable shunting system having a shape memory actuation element, the method comprising:
deploying the adjustable shunting system that incudes the shape memory actuation element at a target location within the patient, wherein the shape memory actuation element is deformed relative to a preferred geometry following deployment of the adjustable shunting system;
percutaneously advancing an energy delivery catheter toward the shunting system until the energy delivery catheter is proximate the adjustable shunting system; and
initiating a power transfer between the energy delivery catheter and the shunting system to induce a current in a resonant circuit that includes the shape memory actuation element,
wherein the current resistively heats the shape memory actuation element.

39. The method of claim 38 wherein the energy delivery catheter is a first catheter, and wherein deploying the adjustable shunting system at the target location includes percutaneously advancing a second catheter different than the first catheter toward the target location, the second catheter carrying the adjustable shunting system.

40. The method of claim 38 wherein percutaneously advancing the energy delivery catheter until the energy delivery catheter is proximate the adjustable shunting system includes positioning a transmitter coil carried by the energy delivery catheter within 5 cm of the adjustable shunting system.

41. The method of claim 40 wherein percutaneously advancing the energy delivery catheter until the energy delivery catheter is proximate the adjustable shunting system includes positioning the transmitter coil within 2 cm of the adjustable shunting system.

42. The method of claim 40 wherein the transmitter coil does not contact the adjustable shunting system.

43. The method of claim 38 wherein the current resistively heats the shape memory actuation element above a transition temperature and causes the shape memory actuation element to move from the deformed configuration toward its preferred geometry.
